(19) United States
(12) Patent Application Publication
(10) Pub. No.: US 2025/0208084 A1
(43) Pub. Date: Jun. 26, 2025

(54) RADIATION-TOLERANT ELECTRODES, POTENTIOMETRIC SENSORS, AND METHODS OF USE

(71) Applicant: Purdue Research Foundation, West Lafayette, IN (US)

(72) Inventors: Alexander Wei, West Lafayette, IN (US); Aiganym Yermembetova, Middletown, DE (US); Bingyuan Zhao, Lafayette, IN (US)

(21) Appl. No.: 18/848,471

(22) PCT Filed: Mar. 31, 2023
(86) PCT No.: PCT/US2023/065227

§ 371 Date: Sep. 18, 2024

Related U.S. Application Data
(60) Provisional application No. 63/326,403, filed on Apr. 1, 2022.

(51) Int. Cl. G01N 27/30; A61L 2/08; G01N 27/327; G01N 27/333

(52) U.S. Cl. CPC G01N 27/302; A61L 2/081; G01N 27/307; G01N 27/308; G01N 27/327; G01N 27/333; A61L 2202/122

(57) ABSTRACT

Radiation-tolerant, thin-film electrodes capable of use in potentiometric sensors and methods of using such electrodes. Such an electrode includes a substrate, an electrode disposed on the substrate, and a protective membrane covering the electrode, wherein the protective membrane is tolerant to gamma radiation such that the protective membrane is capable of tolerating a process of sterilization through gamma radiation.

### BACKGROUND OF THE INVENTION

The present invention generally relates to electrodes, their fabrication, and their use. The invention particularly relates to radiation-tolerant, thin-film electrodes capable of use in potentiometric sensors and methods of using such electrodes.

The bioprocessing industry has been gradually shifting away from stainless-steel bioreactors toward single-use (SU) systems based on thermoplastic materials. Single-use systems avoid potential contamination and circumvent the challenge of re-establishing culture conditions in conventional bioreactors after cleaning and sterilization. Single-use bioreactors typically have plastic bags, tubing, and filters sterilized by gamma (γ) radiation. Such single-use bioreactors have become indispensable in the biopharmaceutical manufacturing of antibodies and other biologics, and more recently for cell- and gene-based therapies that involve the direct infusion of engineered cells into patients. These biomanufacturing technologies require precise monitoring over cell culture conditions for several weeks or more. However, current methods for chemical analysis must be performed offline multiple times using samples extracted from single-use bioreactors, which is tedious and can compromise the sterile environment. Therefore, there is great interest in developing chemical sensors of low size, weight, power, and cost (SWaP-C) that are also compatible with ionizing radiation, to reduce the operational burden and cost of quality control monitoring in single-use systems.

Very few chemical sensors have been validated for in-line monitoring in single-use bioreactors. One type of such validated chemical sensor is encapsulated glass electrodes for pH sensing by potentiometry and dissolved oxygen by fluorescence quenching, which are coupled to single-use systems through custom port adaptors. One example is the OneFerm™ VP 70 pH probe produced by Hamilton, Inc., which can be installed in a standard bioprocess container prior to gamma-ray sterilization, and has a minimum shelf life of twenty-four months. These glass pH probes have been validated in live cell cultures for a 21-day period, then stored in culture media for up to sixty days. The net drift in readings has been reported to be 0.29 pH units over a 21-day period in cell culture and 0.65 pH units over the full 60-day period. Other specifications include pre-calibration accuracy of 0.1 pH with a drift rate of <0.1 pH units/7 days, a dynamic range of pH 3-10, and operating temperatures of 2-50° C.

It is believed that radiation-tolerant SWaP-C sensors for potentiometric sensing or organic thin-film sensors with stable performance after sterilizing radiation have not been previously demonstrated. The development of SWAP-C sensors that can withstand sterilization without compromising function or performance faces various challenges. The sensing modality itself is preferably not be susceptible to defects generated by ionization: the transconductance of ion-selective field-effect transistors (FETs) is adversely affected by trapped charges buried in semiconductor oxides, and photoluminescent dyes are commonly degraded by ionizing radiation, precluding their use in optical sensors. Furthermore, the radiation sensitivity of the transducer element preferably is considered; for example, ionizing radiation is well known to inactivate enzymes and other biomolecules, making them incompatible with sensor sterilization. In fact, many organic materials are prone to damage by radiation or can generate cytotoxic byproducts that leach into cell culture media. The identification of radiation-compatible materials is essentially an empirical process and is the primary reason why radiation-tolerant thin-film sensors have yet to be developed for even the simplest of chemical analytes.

In view of the above, it can be appreciated that it would be desirable if a radiation-tolerant thin-film sensor were available that was capable of overcome one or more of the aforementioned challenges.

### BRIEF SUMMARY OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, radiation-tolerant, thin-film electrodes capable of use in potentiometric sensors and methods of using such electrodes.

According to one nonlimiting aspect of the invention, a radiation-tolerant, thin-film electrode includes a substrate, an electrode disposed on the substrate, and a protective membrane covering the electrode, wherein the protective membrane is tolerant to gamma radiation such that the protective membrane is capable of tolerating a process of sterilization through gamma radiation.

According to another nonlimiting aspect of the invention, the radiation-tolerant, thin-film electrode forms a working electrode including a thermoplastic substrate, a carbon electrode printed on the thermoplastic substrate, a graphene-based solid contact layer disposed on the carbon electrode, and a protective membrane that covers the graphene-based solid contact layer and the carbon electrode. The protective membrane includes an organic matrix of aromatic plasticizer, a miscible polymer, a lipophilic salt, and an ionic receptor.

According to another nonlimiting aspect of the invention, the radiation-tolerant electrode forms a reference electrode wherein the protective membrane includes an underlayer of a polymer film saturated with an inorganic chloride salt and an overlayer that is radiation resistant, seals the underlayer, prevents rapid leaching of the ingredients in the underlayer, and provides ionic conductivity sufficient for stable potentiometry.

According to another nonlimiting aspect of the invention, a potentiometric sensor includes a first radiation-tolerant, thin-film electrode forming a working electrode as disclosed herein, and a second radiation-tolerant electrode forming a reference electrode as disclosed herein.

According to another nonlimiting aspect of the invention, a method of using a thin-film electrode includes sterilizing the thin-film electrode with ionizing irradiation, and obtaining a potentiometric reading that enables pH monitoring with the sterilized thin film electrode.

Technical aspects of radiation-tolerant electrodes, potentiometric sensors, and methods having features as described above preferably include their ability to be used in sterile, single-use systems intended for biomanufacturing or biomedical diagnostics.

Other aspects and advantages will be appreciated from the following detailed description.

### BRIEF DESCRIPTION OF THE DRAWINGS

FIG. 1A is a schematic view of a potentiometric sensor having a working electrode and a reference electrode, and FIG. 1B schematically represents processing steps for fabricating thin-film working and reference electrodes of FIG. 1A according to certain nonlimiting aspects of the invention.

FIG. 2A is a graph showing experimental performance data of γ-irradiated working electrodes of FIG. 1A on day forty-four of a 100-day study (N=8).

FIG. 2B is a graph showing mean changes in voltage per pH unit over a 100-day period with responses defined by the Nernst equation (N=4). Working electrodes of FIG. 1A were paired with a glass reference electrode; readings performed at 25° C.

FIGS. 3A and 3B are graphs showing performance data of electrodes of FIG. 1A over 100 days. FIG. 3A shows data for working electrodes that were γ-irradiated in vacuum-sealed pouches (⁶⁰Co, 25-45 kGy) then conditioned in pH 7 buffer prior to study; the mean voltage drift was <+0.8 mV/day. FIG. 3B shows data for control working electrodes without radiation, with a mean voltage drift<−0.6 mV/day. Working electrodes of FIG. 1A and control working electrodes were paired with a glass reference electrode; readings were performed at 25° C.

FIG. 4 is a graph showing 21-day performance data of electrodes of FIG. 1A (N=4) in cell culture media versus a commercial pH meter (±0.02 pH). Working electrodes of FIG. 1A were paired with a glass reference electrode and calibrated once for voltage drift; differences in readouts vs. pH meter (calibrated daily) are within 0.2 pH units. pH readings were performed at 25° C.

FIG. 5 is a graph showing performance data (inertness) of reference electrodes of FIG. 1A (“y-rad REs”; N=5) and non-irradiated reference electrodes (“Ctrl REs”; N=4) in different pH buffers. Readings from a working electrode (WE) of FIG. 1A are included for comparison. All electrodes were conditioned for three days and paired with a glass reference electrode; readings were performed at 25° C.

### DETAILED DESCRIPTION OF THE INVENTION

The intended purpose of the following detailed description of the invention and the phraseology and terminology employed therein is to describe what is shown in the drawings, which include the depiction of and/or relate to one or more nonlimiting embodiments of the invention, and to describe certain but not all aspects of what is depicted in the drawings, including the embodiment(s) to which the drawings relate. The following detailed description also describes certain investigations relating to the embodiment(s), and identifies certain but not all alternatives of the embodiment(s). As nonlimiting examples, the invention encompasses additional or alternative embodiments in which one or more features or aspects shown and/or described as part of a particular embodiment could be eliminated, and also encompasses additional or alternative embodiments that combine two or more features or aspects shown and/or described as part of different embodiments. Therefore, the appended claims, and not the detailed description, are intended to particularly point out subject matter regarded to be aspects of the invention, including certain but not necessarily all of the aspects and alternatives described in the detailed description.

The present application discloses electrodes, referred to herein as working electrodes and reference electrodes, which can be provided on disposable substrates and include a protective membrane that can be effective in promoting the ability of the electrodes to tolerate sterilizing gamma radiation (γ-rad). The electrodes may be used, for example, as components of a potentiometric sensor capable of use for potentiometric analysis of pH and ions in sterile, single-use systems, as a nonlimiting example, single-use bioreactor systems. Possible materials for the working electrodes include carbon, a graphene-based solid contact layer, and a thin film comprised of an organic matrix of aromatic-rich plasticizer and miscible polymer for structural integrity. This matrix also contains a radiation-compatible organic salt (as a nonlimiting example, a lipophilic salt) for ionic conductivity in a high-impedance system, and a radiation-compatible transducing element (ion receptor) for potentiometric analysis of one or more specific analytes. Possible materials for reference electrodes include printed Ag/AgCl electrodes, though other materials are foreseeable. The working and reference electrodes can be deposited on disposable substrates, for example, printed on a thermoplastic substrate.

In preferred embodiments, the protective membranes are capable of tolerating up to 45 kGy (and possibly higher) of gamma radiation, to enable the potentiometric analysis of pH and ions in sterile environments. Protective membranes have been vetted by the stable performance of working electrodes following irradiation by a ⁶⁰Co source and conditioning before use, with voltage drifts below 1 mV/day over 100 days when paired with a standard glass reference electrode. Embodiments of working electrodes provided with a protective membrane are capable of being stored for at least six months in vacuum-sealed pouches prior to use. Reference electrodes exhibit are capable of exhibiting similar tolerance to γ-radiation and can maintain a constant potential (≤2 mV) between pH 4 and 10. The combined working electrode/reference electrode system provides a Nernstian response to changes in pH or analyte concentrations with a net voltage drift of less than 0.5 mV/day (<0.01 pH units/day), which can be corrected by a single calibration. The data presented hereinafter is based on pH measurements. However, the electrodes (working and reference electrodes) disclosed herein may be used in the potentiometric analysis of aqueous ions in general, as well as various other applications.

The following description describes specific non-limiting example combinations of materials that can be used to fabricate radiation-tolerant potentiometric sensors that are of sufficiently low size, weight, power, and cost (SWaP-C) as to be particularly suitable for use in single-use systems. These examples are supported by experimental demonstrations of pH sensing in biological media under sterile conditions for multiple weeks, and can be extended toward the vetting of potentiometric sensors for ionic analytes in general. Separate formulations are also contemplated for protective membranes suitable for working electrodes and reference electrodes that have different requirements for signal response and stability against voltage drift.

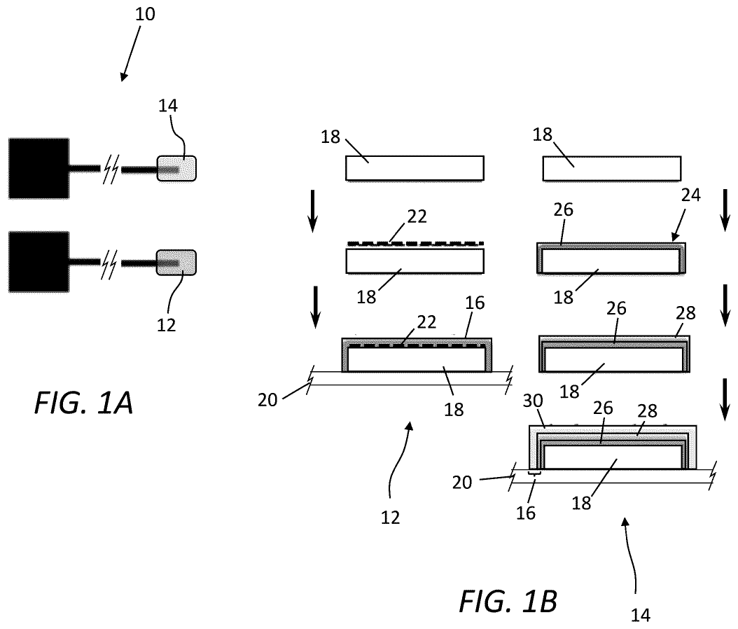

FIG. 1A schematically represents a potentiometric sensor 10 that includes a working electrode 12 and a reference electrode 14 that exemplify certain non-limiting aspects of the invention. Either or both of the working electrode 12 and the reference electrode 14 is preferably a thin-film electrode so as to be flexible and easily shaped to conform to the surface of a substrate 20 that may be formed of a flexible material of various types (e.g., a thermoplastic) and shapes. The potentiometric sensor 10 represented in FIG. 1A is generally configured for monitoring pH.

The electrodes 12 and 14 are each screen-printed electrodes printed on the substrate 20 (which may be the same substrate or different substrates) and represented as being coated with a thin-film protective membrane 16, after which the electrodes 12 and 14 are preferably exposed to γ-radiation prior to their incorporation in their intended application, for example, a sterile single-use system. The width of the active (coated) area of each electrode 12 and 14 can vary widely, for example, a range of at least 1 mm to several centimeters, although smaller and larger areas are foreseeable. The electrodes 12 and 14 preferably have certain beneficial attributes, including being radiation-tolerant and preferably yielding a low SWaP-C sensor 10 that may be disposable. While the electrodes 12 and 14 are referred to herein as “working” electrodes and “reference” electrodes, these terms are used as convenient identifiers and are not intended to otherwise limit the use or structure of the electrodes 12 and 14 or a sensor in which they may be used. For example, the potentiometric sensor 10 may not utilize or require the working electrode 12 or the reference electrode 14 and/or may include more than one of either electrode 12 or 14.

FIGS. 1A and 1B schematically represent the protective membranes 16 as operatively positioned as a functional part of each of the working electrode 12 and the reference electrode 14. According to a preferred aspect of the invention, the membranes 16 on the electrodes 12 and 14 are capable of tolerating the process of sterilization through gamma radiation. In particular, the protective membranes 16 are preferably resistant to gamma radiation such that the membranes 16 are capable of tolerating the process of sterilization through gamma radiation without breaking down or otherwise degenerating so that the working electrode 12 and/or the reference electrode 14 is still functional for their intended purpose, for example, useful for potentiometric analysis of pH or ionic analytes in a sterile, single-use system after having been gamma-irradiated during a gamma-irradiation sterilization process.

The protective membranes 16 are represented in FIG. 1B as formed of one or more membranes or thin films. The protective membranes 16 of the electrodes 12 and 14 represented in FIG. 1B are deposited on, respectively, a carbon electrode 18 and a metallic-covered carbon electrode 24, for example, by being screen-printed on their substrate 20 in roll form. As noted above, the substrate 20 is preferably flexible and may be formed of a plastic, for example, a thermoplastic such as polyethylene terephthalate (PET), although the substrate 20 may be made of or include other materials.

The ability of the potentiometric sensor 10 and its working electrode 12 and reference electrode 14 to suitably withstand sterilizing radiation with minimum loss of function can be quantified. For example, even after having been sterilized by a gamma-irradiation process and stored for several months, the working electrode 12 and reference electrode 14 preferably provide suitable pH sensing capabilities similar to conventional electrodes that are not coated with the protective membrane 16. For example, the working electrode 12 in some embodiments is preferably capable of providing stable readings over 100 days in buffered solutions with a voltage drift of +0.8 m V/day or less, and accurate readings in sterile cell culture media for at least twenty-one days, with readings within 0.2 pH units of a commercial meter. The reference electrode 14 in some embodiments is preferably capable of exhibiting a similar tolerance to gamma radiation and can maintain a constant potential (≤2 mV) between pH 4 and 10.

In addition to the carbon electrode 18, the working electrode 12 is represented in FIG. 1B as including a graphene-based solid contact layer 22 disposed on the carbon electrode 18, over which the protective membrane 16 is then deposited as, in this example, a pH sensing membrane. The protective membrane 16 of the working electrode 12 comprises an organic matrix of an aromatic-rich plasticizer and a miscible polymer for structural integrity, and the matrix also contains small quantities of a radiation-compatible organic salt for ionic conductivity and an ionic receptor for potentiometric analysis of specific analytes. The carbon electrode 18 may be formed on the substrate 20 by any suitable process, such as by a printed process or other deposition process. The protective membrane 16 of the working electrode 12 is shown in FIG. 1B as covering and protectively sealing the electrode 18 and the contact layer 22 from the surrounding environment. The organic matrix of the protective membrane 16 may be a polyvinyl chloride (PVC) and an aromatic-rich plasticizer. A nonlimiting example of a suitable aromatic-rich plasticizer is trioctyl trimellitate (TOTM). A nonlimiting example of a suitable radiation-compatible organic salt is a lipophilic salt, for example, a tetraarylborate salt. The aryl may include a substituted aromatic moiety, and/or a hydrogen- or ion-selective receptor as exemplified herein by the hydrogen ionophore tridodecylamine.

As schematically illustrated in FIG. 1B, the working electrode 16 may be formed by forming the carbon electrode 18 on the substrate 20. Then, the contact layer 22 is deposited on the carbon electrode 18, after which the protective layer 16 is deposited on the contact layer 22 and over the carbon electrode 18 so as to seal the contact layer 22 and the carbon electrode 18 on the substrate 20.

The reference electrode 14 is represented in FIG. 1B as including the metallic-covered electrode 24 on the substrate 20 and the protective layer 16 covering and sealing the electrode 24. The metallic-covered electrode 24 is represented as comprising a metallic layer 26, for example, a layer of Ag/AgCl, printed on a carbon electrode 18, though other materials and types of electrodes are also possible. For example, the electrode 24 may include or be formed of noble metals such as silver, gold, and platinum, and transparent conductors such as indium tin oxide (ITO) and fluorine-doped tin oxide (FTO). The protective membrane 16 of the reference electrode 14 is represented in FIG. 1B as including an underlayer 28 deposited on the electrode 24 and an overlayer 30 overlying the underlayer 28. The underlayer 28 is preferably a thin film of a NaCl-saturated polymer, and the overlayer 30 is preferably a chemically and physically robust polymer layer for adhesion and sealing of the underlayer 28 to prevent rapid leaching of ingredients in the underlayer 28. The underlayer 28 in some embodiments is formed of polyvinylbutryate (PVB) containing an inorganic chloride salt, such as a chloride (Cl)-saturated polyvinyl butyrate (PVB). The overlayer 30 is preferably a radiation-resistant polymer coating. The overlayer 30 preferably provides sufficient ionic conductivity therethrough to allow the reference electrode 14 to function for testing the pH of analytes. The overlayer 30 in some embodiments is formed of an aromatic-rich polyurethane and a PVC/TOTM mixture doped with a small amount of a lipophilic salt. In this example, the underlayer 28 is a chlorine-saturated PVB coated onto the metallic layer 26.

The sensor 10 formed by the combined working electrode/reference electrode system is preferably configured to provide a Nernstian response to changes in pH or concentration of an analyte with a net voltage drift of less than 0.5 mV/day (e.g., <0.01 pH units/day), which is corrected/correctable by a single calibration. In some embodiments, the potentiometric sensor 10, the working electrode 12, and/or the reference electrode 14 can be stored for at least six months in vacuum-sealed pouches prior to use after treatment of said γ-radiation. The thin-film working electrode 12 and reference electrode 14 may be configured for single-use applications and can withstand sterilizing radiation with minimum loss of function. The reference electrode 14 exhibits a tolerance to γ-radiation and can maintain a constant potential (≤2 mV) between pH 4 and 10.

The potentiometric sensor 10, the working electrode 12, and/or the reference electrode 14 electrode may be used to obtain one or more potentiometric readings from a substance in which sterilization is important. In a non-limiting example, a method of using any one or more of the potentiometric sensor 10, the working electrode 12, and/or the reference electrode 14 includes sterilizing the electrode 12 and/or 14 with ionizing irradiation and thereafter obtaining a potentiometric reading that enables pH monitoring with the sterilized electrode 12 and/or 14. The ionizing radiation may be and/or include gamma irradiation, for example as described elsewhere herein. Obtaining the potentiometric reading(s) may include, for example, monitoring pH levels in a biologic media, such as a cell culture, where using sterile instruments is important or even critical. Obtaining the potentiometric reading(s) typically include obtaining a plurality of potentiometric readings across a time span of a day or more, one week or more, or longer for example three weeks or more, as described elsewhere herein. The potentiometric sensor 10, the working electrode 12, and/or the reference electrode 14 electrode may be sealed in a storage container, such as being vacuum packed and sealed in a vacuum sealable thermoplastic pouch, prior to the sterilizing step, and stored in the sealed storage container after the sterilizing step and before the obtaining step for an extended period of time such as a few days, weeks, months, or more, depending on the container characteristic and/or other environmental conditions.

While the potentiometric sensor 10 and the electrodes 12 and 14 are described herein as being used for sensing pH levels in analytes, the inventive concepts behind the potentiometric sensor 10 and/or the electrodes 12 and 14 can be applied toward different types of electrodes, including noble metals such as silver, gold, and platinum, and transparent conductors such as indium tin oxide (ITO) and fluorine-doped tin oxide (FTO).

Next, an illustrative description of a non-limiting example embodiment embodying certain optional, but not necessarily all or necessary, concepts of the invention are provided, as well as findings of certain experiments conducted on the examples.

In this example, the SWAP-C potentiometric sensor 10 includes a working electrode 12 that responds to changes in concentration of specific analytes, and a reference electrode 14 that is essentially inert but can offset voltage drift related to environmental changes. The working electrode 12 and reference electrode 14 are as described in reference to FIG. 1B and formed on a flexible polyethylene terephthalate (PET) roll film. In one illustrative instance, the working electrode 12 comprises the screen-printed carbon electrode 18 coated with an ultrathin layer made of conductive graphene nanoflakes as the contact layer 22 and the protective membrane 16 comprises a plasticizer with an aromatic core, such as trioctyl trimellitate (TOTM), a matrix polymer such as polyvinyl chloride (PVC), a lipophilic base, such as tridodecylamine (TDDA), and a lipophilic anion, such as tetrakis(p-chlorophenyl)borate (TpClB). The reference electrode 14 includes the metallic layer 26, the underlayer 28 formed of polyvinyl butyrate (PVB) saturated with NaCl, and a top layer 30 formed of a material rich in sp² carbon that prevents the depletion of Cl ions while maintaining sufficient ionic conductivity for stable potentiometry. As disclosed herein the concept of “a material rich in sp² carbon” may also be understood as a chemical moiety rich in aromatic rings in a narrower scope, but should be construed with a broader scope than just aromatic rings.

In some illustrative embodiments, the protective membrane 16 may be one or more membranes or thin films, positioned operationally on a disposable electrode as a functional part of the working electrode 12 or reference electrode 14. The membrane 16 for the working electrode 12 is formed of a matrix of polyvinyl chloride (PVC) and aromatic-rich plasticizers such as trioctyl trimellitate (TOTM), a lipophilic salt for ionic conductivity such as tetraarylborate salts where “aryl” refers to substitute aromatic rings, a hydrogen- or ion-selective receptor that is compatible with the formulation described, as demonstrated here by the hydrogen ionophore tridodecylamine. The membrane 16 for the reference electrode 14 may be formed of the underlayer 28 of polyvinylbutryate (PVB) and an inorganic chloride salt such as NaCl, and the overlayer 30 of an aromatic-rich polyurethane and PVC/TOTM mixture doped with a small amount of lipophilic salt.

The membranes 16, (including the under layer 28, and overlayer 30) can be prepared by standard film casting and deposition methods including slot-die coating, Auger pump dispensing, thermal and acoustic dispensing, and piezoelectric inkjet printing. Membrane formulations can be heated or diluted with organic solvents to achieve viscosities appropriate for the chosen method of film deposition, then dried at temperatures that permit full evaporation of the residual solvent. The thin-film electrodes 12 and 14 can then be submitted for sterilization via gamma irradiation between 25 and 45 kGy and stored in vacuum-sealed pouches for at least six months prior to use.

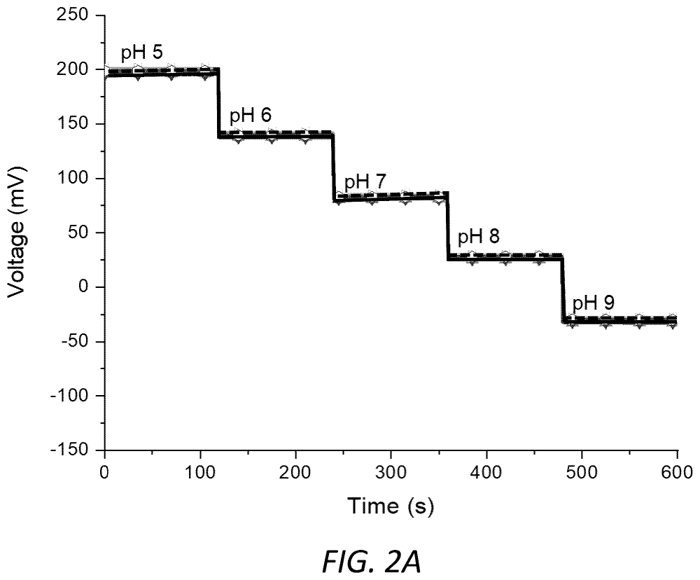

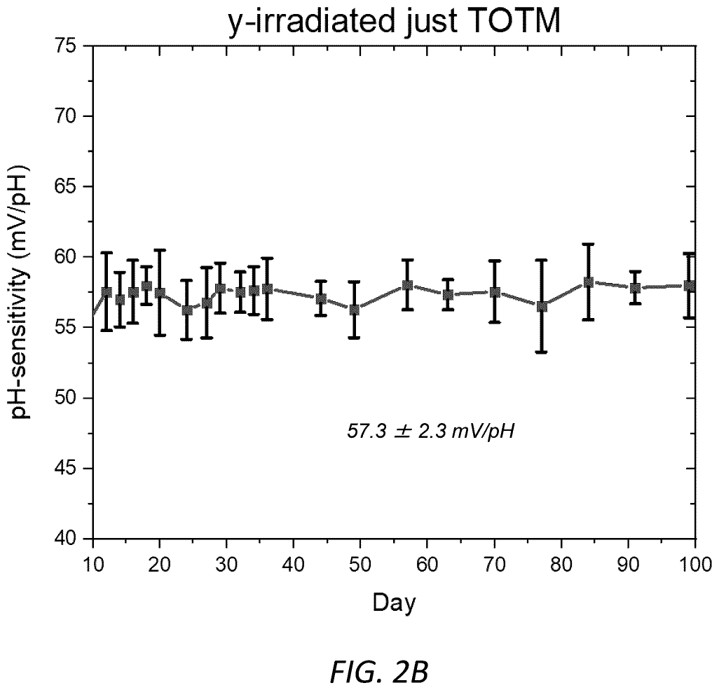

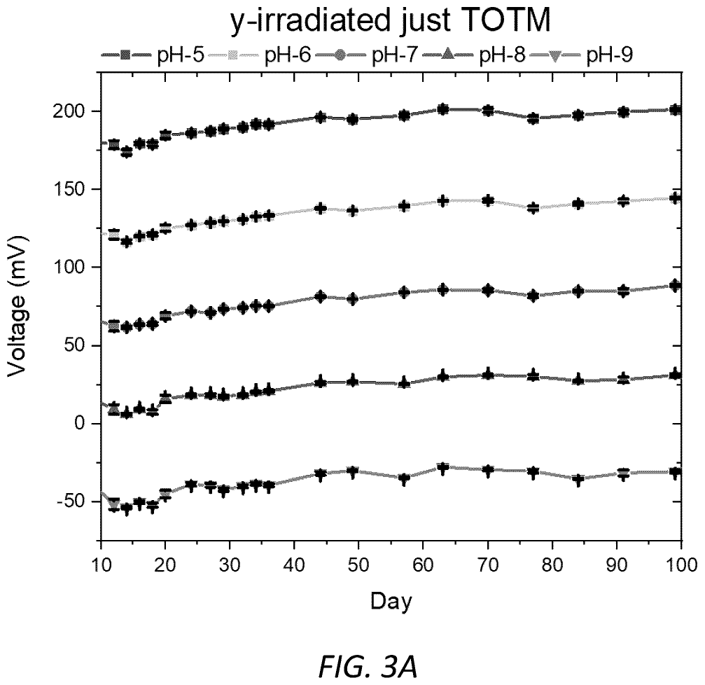

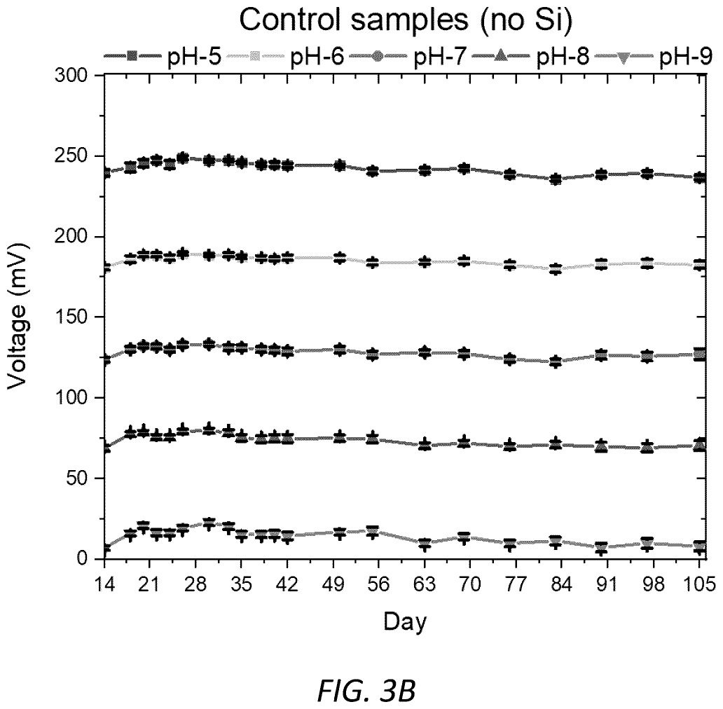

Gamma-irradiated thin-film working electrodes 12 can be connected to standard potentiometric analyzers and used to monitor pH and electrolyte concentrations for many weeks. For example, the pH sensitivity of multiple electrodes was monitored in parallel over the course of three months. Working electrodes 12 conditioned in pH 7 phosphate buffer achieved operational stability (drift≤0.5 mV/h) within 24 hours and full equilibration (drift≤1 mV/day) after 3 days, and produced near-Nernstian responses to changes in acidity between pH 5-9 (57.3 mV/pH unit; see FIGS. 2A and 2B). Voltage readouts were highly reproducible over a 100-day period, with a mean drift rate of +0.8 mV/day (see FIGS. 3A and 3B), or an error of ±0.01 pH. This voltage drift is well below experimental error (±3 mV) as well as the error of a commercial pH meter (±1.2 mV).

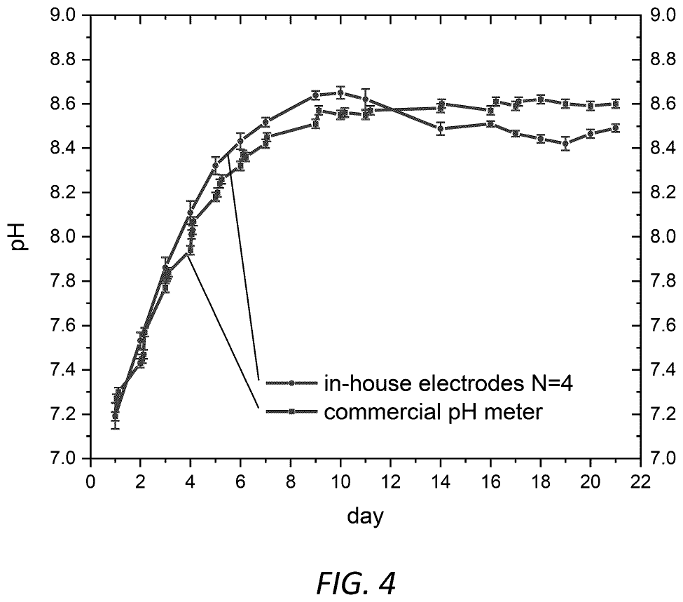

In another example, working electrodes 12 were used to monitor pH changes over a 21-day period in serum-free culture media (EX-CELL CD with 6 mM L-glutamine and 100 μg/mL penicillin/streptomycin). Voltage readings were calibrated once to account for voltage drift, then converted to pH units; a direct comparison against a commercial meter (calibrated daily) showed their readings to be within 0.2 pH units (see FIG. 4). This suggested that the thin-film working electrodes 12 can maintain accuracy for several weeks without corrections between readings.

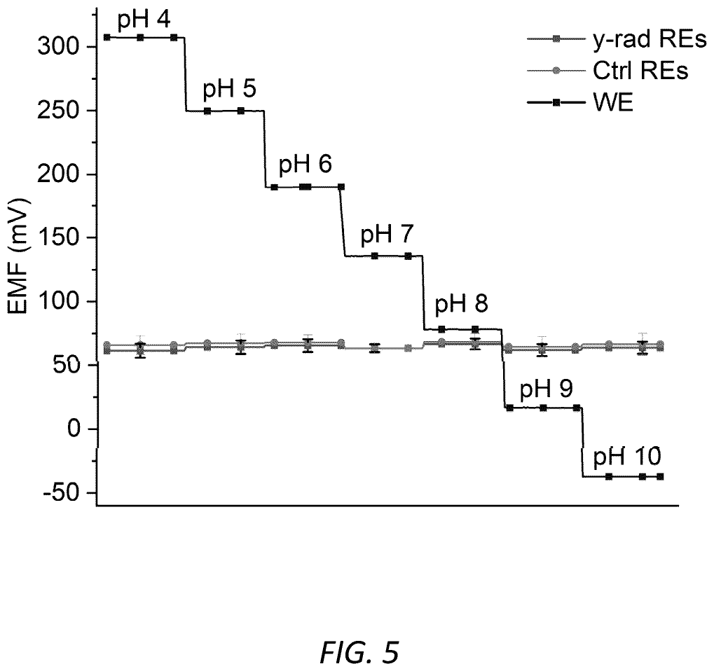

Gamma-irradiated reference electrodes 14 can hold a constant potential for multiple weeks and be paired with working electrodes 12 to provide stable readouts in sterile media. For example, the stability of multiple Ag/AgCl-based reference electrodes were monitored in parallel over the course of 31 days. Reference electrodes conditioned in pH 7 phosphate buffer for 3 days were essentially inert to changes in acidity between pH 4-10, within an error of ±2 mV (see FIG. 5). Thin-film reference electrodes paired with a thin-film working electrode responded to pH changes comparable to that of a commercial pH meter, and the sensor (combined working electrode/reference electrode system) exhibited a mean voltage drift of −0.3 mV/day. This suggested that γ-irradiation effects on thin-film reference electrodes can partially compensate for those on thin-film working electrodes.

Thin-film electrodes 12 and 14 according to some aspects of the invention on substrates 20 can be subjected to γ-radiation (up to 45 kGy) without loss of stability or sensing performance, which is believed to provide important ramifications for monitoring analytes in sterile environments. For example, protective membranes 16 were prepared composed of polyvinyl chloride (PVC), trioctyl trimellitate (TOTM), and a standard hydrogen ionophore were cast onto screen-printed carbon electrodes 18 with exfoliated graphene as the solid contact layer 22. The resulting working electrodes 12 were γ-irradiated and conditioned in phosphate buffers and monitored for up to three months for changes in voltage readout and pH sensitivity, relative to untreated controls. The sensitivities of both the irradiated electrodes 12 and control electrodes were consistently Nernstian over a 100-day window, with both types exhibiting logarithmic voltage decays but in opposite directions. The γ-irradiated working electrodes 12 had excellent long-term stability with quasi-linear voltage drifts of +0.28 mV (˜0.005 pH) per day. Voltage readouts from sterilized thin-film working electrodes 12 in cell culture media could be converted by single-point calibration into pH values that fell within 0.07 units relative to a commercial pH meter (calibrated daily).

Next a detailed description of one example method of producing the electrodes 12 and 14 is provided. It is understood that the electrodes 12 and 14 may be made with different materials, from different suppliers, and/or with other methods. All ingredients for the protective membrane 16 and buffers were obtained in reagent-grade quality. Tetrahydrofuran (THF) was obtained in anhydrous form; aqueous solutions were prepared using deionized water with a measured resistivity above 18 MΩ·cm. pH values were determined using a benchtop meter with daily two-point calibration. Buffers composed of 0.1 M acetic acid/sodium acetate (pH 5-6), 0.1 M disodium hydrogen phosphate (pH 7-8), or 0.1 mM sodium borate (pH 9) were adjusted to their final pH values using 4 M NaOH or 3 M HCl and were replaced every 4-6 weeks. All steps related to thin-film electrode fabrication were conducted in a laboratory fume hood.

Carbon I-bar electrodes with a 2.5″×⅛″ (64×3.2 mm) body and ¼″×¼″ (6.4×6.4 mm) square heads were prepared from carbon paste (DuPont 7102) on 2-mil (50 μm) PET substrates using a Kinzel roll-to-roll screen printer (t_{carbon}=6 μm). Dispersions of graphene nanoplatelets (PureWave, NanoIntegris/Raymor) in dry ethyl acetate (235 μg/mL) were prepared using a mechanochemical process (19) and deposited onto electrode heads with a hand-held airspray gun held at a 45° angle and a distance of 10 cm. Batches of electrodes (N=8) were covered with a plastic mask prior to spray deposition of graphene (5 mL dispersion) and were dried in air prior to membrane coating.

pH-sensing membranes composed of TOTM (65.8 wt. %), PVC (32 wt. %), TDDA (1.6 wt. %), and KtClPhB (0.6 wt. %) were prepared by combining all ingredients in THF (23% w/v) with magnetic stirring at room temperature for 12 h. 100 μL aliquots of membrane cocktail were drop-cast onto graphene-coated electrode heads (6×6 mm2 area) by a micropipette and dried overnight in a glass chamber inside a fume hood, resulting in membranes with a mean thickness of 160 μm as measured by an electronic caliper. The electrodes were packaged in vacuum-sealed thermoplastic pouches using a sealer with membranes protected by a top layer of glassine paper and PET film; they were then shipped to a γ-irradiation provider. Sterilized electrodes were returned 3 weeks after ⁶⁰Co radiation exposure and stored in a dark cabinet for up to 6 months; untreated electrodes from the same batch were vacuum-packed and stored in a similar fashion. Electrodes were removed from the pouches and coated with silicone and then dried for at least 12 h prior to use.

All working electrode and reference electrode materials described above have been vetted previously as membrane components for pH-sensing and reference electrodes, however electrode performance after exposure to γ-radiation from a ⁶⁰Co source (up to 45 kGy) has not been reported. Organic molecules and polymer structures are frequently damaged by exposure to ionizing radiation; common effects include oxidation, chain scission, generation of acid or reactive byproducts, and intermolecular cross linking. Such degradation mechanisms increase the likelihood of changes in chemical and materials properties that can compromise the stable performance and function of potentiometric electrodes. In accordance with some aspects of the present invention, the layered composition of these membranes is capable of tolerating ionizing radiation, the electrodes can be stored for a minimum of 6 months, and provide stable potentiometric readings that enable pH monitoring in cell culture media for a minimum of 3 weeks.

In some arrangements, the potentiometric sensor 10, the working electrode 12, and/or the reference electrode 14 described herein may have a low requirement for sensor calibration. Conventional pH and ion-selective electrodes typically require regular calibration to maintain readout accuracy, but frequent calibration adds operational burden which detracts from the benefits of using disposable, single-use sensors. By demonstrating reproducible readouts with low voltage drift over multiple weeks, the thin-film working electrode 12, and/or the reference electrode 14 described herein may in some configurations reduce the need for regular recalibration and provide a better tradeoff between accuracy and operational simplicity.

Radiation-tolerant sensors with favorable SWAP-C characteristics as disclosed in various forms herein may in some arrangements and/or uses provide benefits to sterile, single-use systems intended for biomanufacturing or biomedical diagnostics. For example, those that are fabricated on flexible, thermoplastic substrates may be attached to the insides of plastic wells or vessels or even integrated directly into the single use system as a form of additive manufacturing. With respect to scalable manufacturing, it is preferably to maintain tight control during membrane deposition for reproducible voltage drift behavior, as the effects of γ-irradiation appears to increase with thickness for manufacturing practical SWAP-C sensors with reliable performance.

Those skilled in the art will recognize that numerous modifications can be made to the specific implementations described above. The implementations should not be limited to the particular limitations described. Other implementations may be possible.

As previously noted above, though the foregoing detailed description describes certain aspects of one or more particular embodiments of the invention, alternatives could be adopted by one skilled in the art. For example, the potentiometric sensor 10, the working electrode 12, and/or the reference electrode 14 and their components, could differ in appearance and construction from the embodiments described herein and shown in the drawings, functions of certain components of the potentiometric sensor 10, the working electrode 12, and/or the reference electrode 14 could be performed by components of different construction but capable of a similar (though not necessarily equivalent) function, and various materials could be used in the fabrication of the potentiometric sensor 10, the working electrode 12, and/or the reference electrode 14 and/or their components. As such, and again as was previously noted, it should be understood that the invention is not necessarily limited to any particular embodiment described herein or illustrated in the drawings.

1. A radiation-tolerant, thin-film electrode for a potentiometric sensor, the electrode comprising:
a substrate;
an electrode disposed on the substrate; and
a protective membrane covering the electrode, wherein the protective membrane is tolerant to gamma radiation such that the protective membrane is capable of tolerating a process of sterilization through gamma radiation, to enable the potentiometric analysis of pH and ions in sterile environments.

2. The radiation-tolerant, thin-film electrode of claim 1, wherein the radiation-tolerant electrode comprises a working electrode:
wherein the substrate is a thermoplastic substrate;
wherein the electrode comprises a carbon electrode printed on the thermoplastic substrate; and
a graphene-based solid contact layer disposed on the carbon electrode,
wherein the protective membrane covers the graphene-based solid contact layer and the carbon electrode, the protective membrane comprising an organic matrix of aromatic plasticizer, a miscible polymer, a lipophilic salt, and an ionic receptor.

3. The radiation-tolerant, thin-film electrode of claim 2 comprising a working electrode,
wherein the ionic receptor comprises a radiation-compatible organic salt for ionic conductivity in a high-impedance system, and
wherein the lipophilic salt comprises a radiation-compatible ion receptor for potentiometric analysis of a specific analyte.

4. The radiation-tolerant, thin-film electrode of claim 2 comprising a working electrode, wherein the aromatic-rich plasticizer comprises trioctyl trimellitate (TOTM) and said lipophilic salt for ionic conductivity comprises a tetraarylborate salt.

5. The radiation-tolerant, thin-film electrode of claim 1, wherein the radiation-tolerant electrode comprises a reference electrode, wherein the protective membrane covers the electrode, the protective membrane comprising:
an underlayer comprising a polymer film saturated with an inorganic chloride salt; and
an overlayer that is radiation resistant, seals the underlayer, prevents rapid leaching of the ingredients in the underlayer, and provides ionic conductivity sufficient for stable potentiometry.

6. The radiation-tolerant, thin-film electrode of claim 5 comprising a reference electrode, wherein the electrode comprises an Ag/AgCl electrode, the underlayer comprises a NaCl-saturated polymer membrane, and the overlayer comprises an aromatic-rich polymer.

7. The radiation-tolerant, thin-film electrode of claim 6 comprising a reference electrode, wherein the polymer film of the underlayer comprises polyvinylbutryate (PVB).

8. The radiation-tolerant, thin-film electrode of claim 5 comprising a reference electrode, wherein the underlayer comprises a chloride-saturated polyvinyl butyrate (PVB), and the overlayer comprises a radiation-resistant polymer coating for adhesion and sealing of the chloride-saturated PVB.

9. The radiation-tolerant, thin-film electrode of claim 5 comprising a reference electrode, wherein the overlayer comprises an aromatic-rich polyurethane and a PVC/TOTM mixture doped with a lipophilic salt.

10. A potentiometric sensor comprising:
a first radiation-tolerant, thin-film electrode according to claim 1, wherein the first radiation-tolerant electrode is a working electrode; and
a second radiation-tolerant electrode, thin-film according to claim 1, wherein the first radiation-tolerant electrode is a reference electrode.

11. The potentiometric sensor of claim 10, wherein the potentiometric sensor provides a Nernstian response to changes in pH or concentration of an analyte with a net voltage drift of less than 0.5 mV/day (e.g., <0.01 pH units/day), which is corrected/correctable by a single calibration.

12. The potentiometric sensor of claim 10, wherein the working electrode and/or reference electrode can be stored for at least 6 months in vacuum-sealed pouches prior to use after the process of sterilization through gamma radiation.

13. The potentiometric sensor of claim 10, wherein the working electrode and the reference electrode can withstand sterilizing radiation with minimum loss of function.

14. A method of using the thin-film electrode of claim 1, the method comprising:
sterilizing the thin-film electrode with ionizing irradiation; and
obtaining a potentiometric reading that enables pH monitoring with the sterilized thin film electrode.

15. The method of claim 14, wherein the ionizing radiation comprises gamma irradiation.

16. The method of claim 14, wherein the step of obtaining a potentiometric reading comprises monitoring pH levels in a biologic media.

17. The method of claim 16, wherein the biologic media comprises a cell culture

18. The method of claim 16, wherein the step of obtaining a potentiometric reading comprises obtaining a plurality of potentiometric readings across a time span of one week or more.

19. The method of claim 14, further comprising:
sealing the thin-film electrode in a storage container prior to the sterilizing step; and
storing the thin-film electrode in the sealed storage container after the sterilizing step and before the obtaining step.

20. The method of claim 19, wherein the storage container comprises a vacuum sealable pouch, and wherein the step of sealing includes vacuum sealing the storage container.
